(19) United States
(12) Patent Application Publication
(10) Pub. No.: US 2023/0029466 A1
(43) Pub. Date: Feb. 2, 2023

(54) Pocketed Compression Sheath

(71) Applicants: WILLIAM SANDERS, GARNER, NC (US); Kizzy Sanders, GARNER, NC (US)

(72) Inventors: WILLIAM SANDERS, GARNER, NC (US); Kizzy Sanders, GARNER, NC (US)

(21) Appl. No.: 17/443,581

(22) Filed: Jul. 27, 2021

(51) Int. Cl. A61F 5/01; A61F 7/02

(52) U.S. Cl. CPC A61F 5/0109; A61F 5/0111; A61F 7/02; A61F 2007/0042

(57) ABSTRACT

The Pocketed Compression sheath is polyester compressive material formed into an open-ended sheath. Squares of the same type of polyester material are sewn onto the sides of the open-ended sheath to form pockets. The sheath is intended to be slid onto an extremity such as foot. Figure B illustrates a larger version intended to be utilized for the mid to upper leg. The pockets are intended to be place holders for heating packs or ice packs to help minimize swelling and pain in muscles and joints.

### CROSS-REFERENCE TO RELATED APPLICATIONS

N/A

### BACKGROUND OF THE INVENTION

In the field of athletics and in daily life if an individual sustains a minor injury to an extremity such as a hand or foot, one is regulated to applying ice. When applying the ice, one would have to bag ice and hold it in place by hand or using large amounts of wrap to hold in place. Our invention utilizes pockets to hold the heating or cooling element in place while also using compression to minimize swelling and soreness.

### BRIEF SUMMARY OF THE INVENTION

The purpose is to combine compression therapy, cryotherapy or thermotherapy in a form factor that is convenient for an individual to apply without the need to wrap to an extremity.

### DESCRIPTION

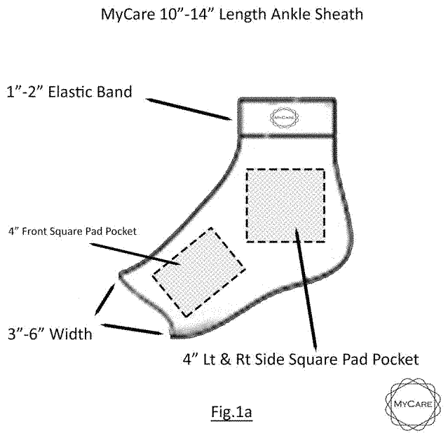

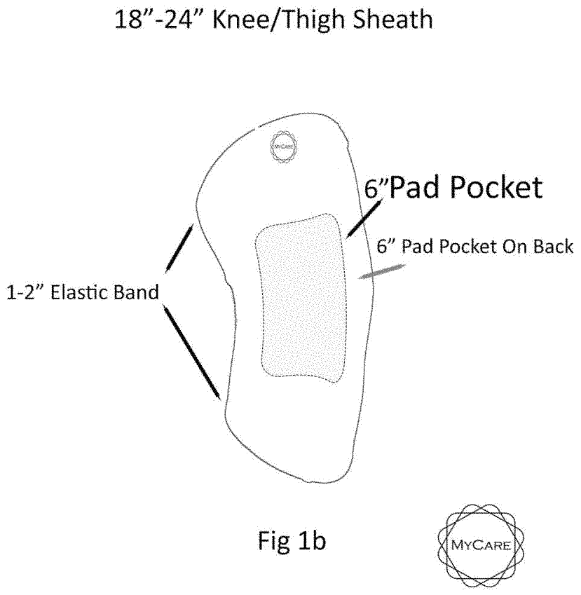

FIG. 1a. The “Pocketed Compression Sheath” is a 100% polyester, elastic material sewn into the form of a ten-to-fourteen-inch compressive sheath that can used to cover the body's extremities i.e., foot and areas of the body that tend to have tendonitis and soreness. The utility patent submission is for the four-to-six-inch square or rectangular pockets/holders that are sewn onto the sheath to hold cold pads, heating pads and bagged ice. Three out of four sides of the 4-6″ added square or rectangular material is sewn onto the sheath to create a pocket of holding space. The “pocket” is also made of 100% polyester, elastic material. The cold or heat treatment transfers through the material to help alleviate soreness and inflammation. The compressive material also helps in alleviating inflammation.

### Description of FIG. 1b

FIG. 1b. The “Pocketed Compression Sheath” is a 100% polyester, elastic material sewn into the form of an eighteen-to-twenty-four-inch compressive sheath that can used to cover the body's extremities i.e., foot and areas of the body that tend to have tendonitis and soreness. The utility patent submission is for the six-inch square or rectangular pockets/holders that are sewn onto the sheath to hold cold pads, heating pads and bagged ice. Two 6″ added squares or rectangular material is sewn onto the sheath to create a pocket of holding space intended to lie on the knee and on the back of the knee. The “pocket” is also made of 100% polyester, elastic material. The cold or heat treatment transfers through the material to help alleviate soreness and inflammation. The compressive material also helps in alleviating inflammation.

### DETAILED DESCRIPTION OF THE INVENTION

Process of sewing a 14″×6″ polyester, compressive material into a sheath by joining together the left and right ends of the material. Two to three 4″ or 6″ square pieces of like material are also sewn onto the initial piece of material that formed the sheath. The additional smaller pieces are sewn on the left, right and bottom of the square to leave the top of the square open to form a pocket to allow insertion of the cooling or heating element. There are variations in size to accommodate a larger extremity such as the knee and thigh as illustrated in FIGS. 1a and 1b.

1. I, William Sanders, one of the named inventors is hereby submitting a claim to be filed with the patent application Ser. No. 17/443,581 for the “Pocketed Compression Sheath”. The “Pocketed Compression Sheath” is a 100% polyester, elastic material sewn into the form of a ten-to-fourteen-inch compressive sheath that can used to cover the body's extremities i.e., foot and areas of the body that tend to have tendonitis, swelling and soreness. Longer, larger part of an extremity such as the mid to upper leg will require a larger sheath at approximately eighteen to twenty-four inches. The utility patent submission is for the four-to-six-inch square or rectangular pockets/holders that are sewn onto the sheath to hold cold pads, heating pads and bagged ice. Three out four sides of the 4-6″ added square or rectangular material is sewn onto the sheath to create a pocket of holding space. The “pocket” is also made of 100% polyester, elastic material. The cold or heat treatment transfers through the material to help alleviate soreness and inflammation. The compressive material also helps in alleviating inflammation.
